(19) United States
(12) Patent Application Publication
(10) Pub. No.: US 2015/0250407 A1
(43) Pub. Date: Sep. 10, 2015

(54) BREATH ANALYZER AND BREATH TEST METHODS

(71) Applicant: Anastasia Rigas, Setauket, NY (US)

(72) Inventor: Anastasia Rigas, Setauket, NY (US)

(21) Appl. No.: 14/719,794

(22) Filed: May 22, 2015

Related U.S. Application Data
(63) Continuation-in-part of application No. 13/420,321, filed on Mar. 14, 2012.

(51) Int. Cl. A61B 5/08; G01N 33/00; A61B 5/00; A61B 5/097

(52) U.S. Cl. CPC A61B 5/082; A61B 5/097; G01N 33/005; A61B 5/4255; A61B 5/486; A61B 5/7275; A61B 5/7282; A61B 5/4866; A61B 5/7246; A61B 2560/0214

(57) ABSTRACT

The present invention provides an improved breath analyzer and breath test method to determine the presence of a gastrointestinal disorder in a subject's digestive tract.

### PRIORITY

This application is a continuation-in-part of U.S. utility application Ser. No. 13/420,321 and also claims priority to U.S. provisional application Nos. 62/001,832 and 62/013,133, each of which are herein incorporated by reference.

### FIELD

The present application relates generally to a breath analyzer and a breath test method for detecting hydrogen in breath to determine presence of a gastrointestinal disorder in a subject's digestive tract.

### BACKGROUND

Gastrointestinal disorders are known. Celiac disease is an immune mediated enteropathy caused by ingestion of gluten, a protein found in wheat, and affects people who are genetically predisposed. Non-celiac gluten sensitivity (NCGS) is caused by the ingestion of gluten as well. Lactose intolerance is caused because of inability to digest lactose, a carbohydrate contained in dairy products, due to lack or decreased production of the enzyme lactase by the intestine. Fructose intolerance is caused by the inability to digest fructose, a carbohydrate found in fruits, due to lack or decreased production of the enzyme aldolase B by the intestine. Small intestinal bacterial overgrowth (SIBO) is caused by abnormally increased number of bacteria in the small bowel.

Current methods for diagnosing these gastrointestinal disorders have drawbacks. It would be desirable to provide easy, non-invasive and reliable point-of-care device and test to determine presence of celiac disease, NCGS, lactose intolerance, fructose intolerance and/or SIBO. It would also be desirable to provide a device and test that is less costly, less cumbersome and more convenient than existing tests.

### BRIEF DESCRIPTION OF DRAWINGS

The following drawings are illustrative of particular examples of the present invention and therefore do not limit the scope of the invention. The drawings are not to scale (unless so stated) and are intended for use in conjunction with the explanations in the following detailed description. Examples of the present invention will hereinafter be described in conjunction with the appended drawings, wherein like numerals denote like elements.

FIG. 1 illustrates an acid-base (emeraldine salt (ES)-emeraldine base (EB)) transition for polyaniline;

FIG. 2 shows an embodiment of a hydrogen sensor;

FIG. 3 shows a schematic of an embodiment of a breath analyzer;

FIG. 4 shows an embodiment of a breath analyzer with a removable mouthpiece and a main body in a detached configuration;

FIG. 5 shows an embodiment of a breath analyzer with a removable mouthpiece and a main body in an attached configuration;

FIG. 6 shows an embodiment of an electrical schematic for the breath analyzer;

FIG. 7 shows an embodiment of a desiccant assembly; and

FIG. 8 shows another embodiment of a desiccant assembly.

### SUMMARY

The present invention provides a breath analyzer and breath test method to determine the presence of hydrogen in a subject's digestive tract. Subjects afflicted either by celiac disease, non-celiac gluten sensitivity, lactose intolerance, fructose intolerance or small bowel bacterial overgrowth have a higher level of hydrogen in their breath at varying degrees depending on which ailment they carry.

Some embodiments provide a portable, hand-held breath analyzer including a main body and a removable mouthpiece, wherein the removable mouthpiece removably attaches to the main body. The main body includes sensor, a processor, a power source and an electrical circuit. The electrical circuit operably connects the power source to the sensor and connects the sensor to the processor. The sensor comprises a hydrogen selective material and a conductive material, wherein the hydrogen selective material contacts the conductive material. The hydrogen selective material has a resistivity that increases in response to increased concentration of hydrogen. The processor detects resistivity of the sensor and uses the resistivity to calculate a concentration of hydrogen in a breath sample.

In some cases, the hydrogen selective material comprises polyaniline, wherein the polyaniline is doped with a dopant that increases pH sensitivity of the polyaniline, and wherein the polyaniline has a resistivity that increases in response to increased concentration of hydrogen. In certain cases, the polyaniline has a pH sensitivity of more than 59 mV. The dopant can include a protonic acid in some embodiments, such as a protonic acid selected from the group consisting of hydrochloric acid, sulfuric acid, salicylic acid, acetic acid, citric acid, tartaric acid, oxalic acid, malonic acid, succinic acid, glutamic acid, adipic acid, phthalic acid and camphor sulfonic acid. In certain cases, the dopant comprises hydrochloric acid.

In some cases, the conductive material comprises plurality of electrodes. The plurality of electrodes can have any desired arrangement, such as a plurality of interdigitated finger electrodes. Also, in some cases, hydrogen selective material is deposited as a thin film on the conductive material.

Some embodiments provide a breath test method for screening for a gastrointestinal disorder. The screening method includes providing a portable, hand-held breath analyzer, prompting a subject to exhale a breath sample into a removable mouthpiece, allowing the processor to measure a resistivity of a sensor that occurs when the breath sample contacts the sensor, and designating the subject as having an increased likelihood of having a gastrointestinal disorder if the measured resistivity is above and/or beneath a predetermined value. In some cases, the gastrointestinal disorder is celiac disease. In other cases, the gastrointestinal disorder is non-celiac gluten sensitivity.

Other embodiments provide a breath test method for diagnosing a gastrointestinal disorder. The diagnostic method includes providing a portable, hand-held breath analyzer, prompting a subject to exhale a breath sample into a removable mouthpiece, allowing the processor to measure a resistivity of a sensor that occurs when the breath sample contacts the sensor, and diagnosing the subject as having a gastrointestinal disorder if the measured resistivity is above and/or beneath a predetermined value. In some cases, the gastrointestinal disorder is celiac disease. In other cases, the gastrointestinal disorder is non-celiac gluten sensitivity.

Other embodiments provide a breath test method for screening for lactose intolerance. The screening method includes providing a portable, hand-held breath analyzer, prompting a subject to exhale a baseline breath sample into a removable mouthpiece, allowing the processor to measure a resistivity of a sensor that occurs when the baseline breath sample contacts the sensor, prompting a subject to ingest a lactose-containing meal, prompting a subject to exhale a post-lactose breath sample into the removable mouthpiece, allowing the processor to measure a resistivity of the sensor that occurs when the post-lactose breath sample contacts the sensor, comparing the measured resistivity of the baseline breath sample to the measured resistivity of the post-lactose breath sample, and designating the subject as having an increased likelihood of having lactose intolerance if the comparing meets a predetermined criteria. In some cases, the designating the subject as having an increased likelihood of having lactose intolerance occurs when the measured resistivity is above and/or beneath a predetermined value.

In some embodiments, the breath test method for screening for lactose intolerance includes prompting a subject to exhale a first post-lactose breath sample into the removable mouthpiece at a first time point, allowing the processor to measure a resistivity of the sensor that occurs when the first post-lactose breath sample contacts the sensor, prompting a subject to exhale a second post-lactose breath sample into the removable mouthpiece at a second time point, allowing the processor to measure a resistivity of the sensor that occurs when the second post-lactose breath sample contacts the sensor, comparing the measured resistivity of the baseline breath sample, the first post-lactose breath sample and the second post-lactose breath sample, and designating the subject as having an increased likelihood of having lactose intolerance if the comparing meets a predetermined criteria.

In other embodiments, the breath test method for screening for lactose intolerance includes prompting a subject to exhale a first post-lactose breath sample into the removable mouthpiece at a first time point, allowing the processor to measure a resistivity of the sensor that occurs when the first post-lactose breath sample contacts the sensor, prompting a subject to exhale a second post-lactose breath sample into the removable mouthpiece at a second time point, allowing the processor to measure a resistivity of the sensor that occurs when the second post-lactose breath sample contacts the sensor, prompting a subject to exhale a third post-lactose breath sample into the removable mouthpiece at a third time point, allowing the processor to measure a resistivity of the sensor that occurs when the third post-lactose breath sample contacts the sensor, prompting a subject to exhale a fourth post-lactose breath sample into the removable mouthpiece at a fourth time point, allowing the processor to measure a resistivity of the sensor that occurs when the fourth post-lactose breath sample contacts the sensor, comparing the measured resistivity of the baseline breath sample, the first post-lactose breath sample, the second post-lactose breath sample, the third post-lactose breath sample and the fourth post-lactose breath sample, and designating the subject as having an increased likelihood of having lactose intolerance if the comparing meets a predetermined criteria. In some cases, the first time point is a 30 minute time point, the second time point is a 60 minute time point, the third time point is a 90 minute time point and the fourth time point is a 120 minute time point.

Other embodiments provide a breath test method for diagnosing lactose intolerance. The screening method includes providing a portable, hand-held breath analyzer, prompting a subject to exhale a baseline breath sample into a removable mouthpiece, allowing the processor to measure a resistivity of a sensor that occurs when the baseline breath sample contacts the sensor, prompting a subject to ingest a lactose-containing meal, prompting a subject to exhale a post-lactose breath sample into the removable mouthpiece, allowing the processor to measure a resistivity of the sensor that occurs when the post-lactose breath sample contacts the sensor, comparing the measured resistivity of the baseline breath sample to the measured resistivity of the post-lactose breath sample, and diagnosing the subject with lactose intolerance if the comparing meets a predetermined criteria. In some cases, the diagnosing the subject with lactose intolerance occurs when the measured resistivity is above and/or beneath a predetermined value.

In some embodiments, the breath test method for diagnosing lactose intolerance includes prompting a subject to exhale a first post-lactose breath sample into the removable mouthpiece at a first time point, allowing the processor to measure a resistivity of the sensor that occurs when the first post-lactose breath sample contacts the sensor, prompting a subject to exhale a second post-lactose breath sample into the removable mouthpiece at a second time point, allowing the processor to measure a resistivity of the sensor that occurs when the second post-lactose breath sample contacts the sensor, comparing the measured resistivity of the baseline breath sample, the first post-lactose breath sample and the second post-lactose breath sample, and diagnosing the subject with lactose intolerance if the comparing meets a predetermined criteria.

In other embodiments, the breath test method for diagnosing lactose intolerance includes prompting a subject to exhale a first post-lactose breath sample into the removable mouthpiece at a first time point, allowing the processor to measure a resistivity of the sensor that occurs when the first post-lactose breath sample contacts the sensor, prompting a subject to exhale a second post-lactose breath sample into the removable mouthpiece at a second time point, allowing the processor to measure a resistivity of the sensor that occurs when the second post-lactose breath sample contacts the sensor, prompting a subject to exhale a third post-lactose breath sample into the removable mouthpiece at a third time point, allowing the processor to measure a resistivity of the sensor that occurs when the third post-lactose breath sample contacts the sensor, prompting a subject to exhale a fourth post-lactose breath sample into the removable mouthpiece at a fourth time point, allowing the processor to measure a resistivity of the sensor that occurs when the fourth post-lactose breath sample contacts the sensor, comparing the measured resistivity of the baseline breath sample, the first post-lactose breath sample, the second post-lactose breath sample, the third post-lactose breath sample and the fourth post-lactose breath sample; and diagnosing the subject with lactose intolerance if the comparing meets a predetermined criteria. In some cases, the first time point is a 30 minute time point, the second time point is a 60 minute time point, the third time point is a 90 minute time point and the fourth time point is a 120 minute time point.

Other embodiments provide a breath test method for screening for fructose intolerance. The screening method includes providing a portable, hand-held breath analyzer, prompting a subject to exhale a baseline breath sample into a removable mouthpiece, allowing the processor to measure a resistivity of a sensor that occurs when the baseline breath sample contacts the sensor, prompting a subject to ingest a fructose-containing meal, prompting a subject to exhale a post-fructose breath sample into the removable mouthpiece, allowing the processor to measure a resistivity of the sensor that occurs when the post-fructose breath sample contacts the sensor, comparing the measured resistivity of the baseline breath sample to the measured resistivity of the post-fructose breath sample, and designating the subject as having an increased likelihood of having fructose intolerance if the comparing meets a predetermined criteria. In some cases, the designating the subject as having an increased likelihood of having fructose intolerance occurs when the measured resistivity is above and/or beneath a predetermined value.

In some embodiments, the breath test method for screening for fructose intolerance includes prompting a subject to exhale a first post-fructose breath sample into the removable mouthpiece at a first time point, allowing the processor to measure a resistivity of the sensor that occurs when the first post-fructose breath sample contacts the sensor, prompting a subject to exhale a second post-fructose breath sample into the removable mouthpiece at a second time point, allowing the processor to measure a resistivity of the sensor that occurs when the second post-fructose breath sample contacts the sensor, comparing the measured resistivity of the baseline breath sample, the first post-fructose breath sample and the second post-fructose breath sample, and designating the subject as having an increased likelihood of having fructose intolerance if the comparing meets a predetermined criteria.

In other embodiments, the breath test method for screening for fructose intolerance includes prompting a subject to exhale a first post-fructose breath sample into the removable mouthpiece at a first time point, allowing the processor to measure a resistivity of the sensor that occurs when the first post-fructose breath sample contacts the sensor, prompting a subject to exhale a second post-fructose breath sample into the removable mouthpiece at a second time point, allowing the processor to measure a resistivity of the sensor that occurs when the second post-fructose breath sample contacts the sensor, prompting a subject to exhale a third post-fructose breath sample into the removable mouthpiece at a third time point, allowing the processor to measure a resistivity of the sensor that occurs when the third post-fructose breath sample contacts the sensor, prompting a subject to exhale a fourth post-fructose breath sample into the removable mouthpiece at a fourth time point, allowing the processor to measure a resistivity of the sensor that occurs when the fourth post-fructose breath sample contacts the sensor, comparing the measured resistivity of the baseline breath sample, the first post-fructose breath sample, the second post-fructose breath sample, the third post-fructose breath sample and the fourth post-fructose breath sample; and designating the subject as having an increased likelihood of having fructose intolerance if the comparing meets a predetermined criteria. In some cases, the first time point is a 30 minute time point, the second time point is a 60 minute time point, the third time point is a 90 minute time point and the fourth time point is a 120 minute time point.

Other embodiments provide a breath test method for diagnosing fructose intolerance. The screening method includes providing a portable, hand-held breath analyzer, prompting a subject to exhale a baseline breath sample into a removable mouthpiece, allowing the processor to measure a resistivity of a sensor that occurs when the baseline breath sample contacts the sensor, prompting a subject to ingest a fructose-containing meal, prompting a subject to exhale a post-fructose breath sample into the removable mouthpiece, allowing the processor to measure a resistivity of the sensor that occurs when the post-fructose breath sample contacts the sensor, comparing the measured resistivity of the baseline breath sample to the measured resistivity of the post-fructose breath sample, and diagnosing the subject with fructose intolerance if the comparing meets a predetermined criteria. In some cases, the diagnosing the subject with fructose intolerance occurs when the measured resistivity is above and/or beneath a predetermined value.

In some embodiments, the breath test method for diagnosing fructose intolerance includes prompting a subject to exhale a first post-fructose breath sample into the removable mouthpiece at a first time point, allowing the processor to measure a resistivity of the sensor that occurs when the first post-fructose breath sample contacts the sensor, prompting a subject to exhale a second post-fructose breath sample into the removable mouthpiece at a second time point, allowing the processor to measure a resistivity of the sensor that occurs when the second post-fructose breath sample contacts the sensor, comparing the measured resistivity of the baseline breath sample, the first post-fructose breath sample and the second post-fructose breath sample, and diagnosing the subject with fructose intolerance if the comparing meets a predetermined criteria.

In other embodiments, the breath test method for diagnosing fructose intolerance includes prompting a subject to exhale a first post-fructose breath sample into the removable mouthpiece at a first time point, allowing the processor to measure a resistivity of the sensor that occurs when the first post-fructose breath sample contacts the sensor, prompting a subject to exhale a second post-fructose breath sample into the removable mouthpiece at a second time point, allowing the processor to measure a resistivity of the sensor that occurs when the second post-fructose breath sample contacts the sensor, prompting a subject to exhale a third post-fructose breath sample into the removable mouthpiece at a third time point, allowing the processor to measure a resistivity of the sensor that occurs when the third post-fructose breath sample contacts the sensor, prompting a subject to exhale a fourth post-fructose breath sample into the removable mouthpiece at a fourth time point, allowing the processor to measure a resistivity of the sensor that occurs when the fourth post-fructose breath sample contacts the sensor, comparing the measured resistivity of the baseline breath sample, the first post-fructose breath sample, the second post-fructose breath sample, the third post-fructose breath sample and the fourth post-fructose breath sample; and diagnosing the subject with fructose intolerance if the comparing meets a predetermined criteria. In some cases, the first time point is a 30 minute time point, the second time point is a 60 minute time point, the third time point is a 90 minute time point and the fourth time point is a 120 minute time point.

### DETAILED DESCRIPTION

The following detailed description is exemplary in nature and is not intended to limit the scope, applicability, or configuration of the invention in any way. Rather, the following description provides some practical illustrations for implementing examples of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives. For example, each time the term “comprising” is used, in alternative embodiments, “comprising” can be replaced with “consisting essentially of” or “consisting of.”

The present invention provides an improved breath analyzer and breath test method to determine the presence of a gastrointestinal disorder (e.g., celiac disease, non-celiac gluten sensitivity, lactose intolerance, fructose intolerance or small bowel bacterial overgrowth) in a subject's digestive tract. The improved breath analyzer and breath test is less costly and more convenient than existing methods.

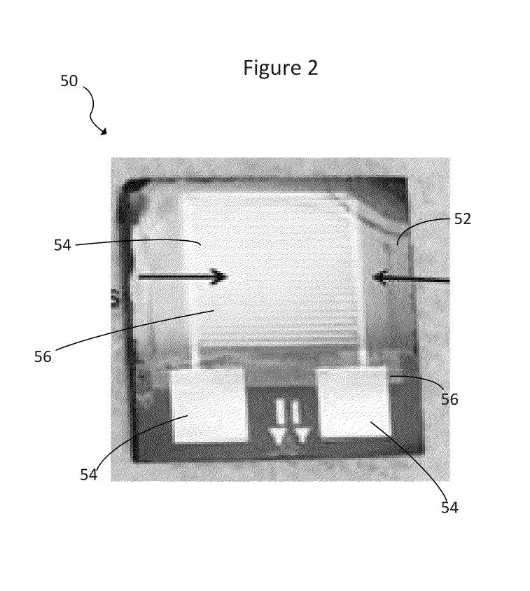

FIG. 2 illustrates an exemplary embodiment of a hydrogen detecting sensor 50. The hydrogen detecting sensor 50 includes a substrate 52, a conductive material 54 and hydrogen selective material 56. The conductive material 54 is provided on the substrate 52 and the hydrogen selective material 56 contacts the conductive material 54.

The conductive material 54 includes any desired conductive material. In some cases, the conductive material 54 is platinum. In other cases, the conductive material 54 is gold. In certain cases, the conductive material 54 is an electrode arrangement. The electrode arrangement can be a single electrode or a plurality of electrodes. The electrodes can be spaced apart in any desired arrangement. In some cases, the electrodes are spaced less than about 250 μm apart, perhaps less than about 150 μm apart, such as about 100 μm apart. In certain cases, the electrodes are spaced less than about 10 μm apart, such as 5 μm apart. In some cases, the electrodes include interdigitated finger electrodes. In one embodiment, the sensor comprises interdigitated platinum finger electrodes with a line spacing of 100 μm apart or less. In the embodiment of FIG. 2, the conductive material 54 is provided as an arrangement of interdigitated finger electrodes.

The hydrogen selective material 56 includes any material that has a resistivity that increases in response to increased presence of hydrogen. In some cases, the hydrogen selective material 56 is a hydrogen selective metal oxide. In certain cases, the hydrogen selective material 56 comprises a hydrogen selective zinc oxide. In other cases, the hydrogen selective material 56 comprises a hydrogen selective metal oxide film. In particular cases, the hydrogen selective material 56 comprises a hydrogen selective metal zinc oxide film. In other cases, the hydrogen selective material 56 comprises zinc oxide nanowires. In certain cases, the hydrogen selective material 56 comprises a film comprising zinc oxide nanowires. In some cases, the zinc oxide nanowires can have a diameter of 200 nm or less, such as 100 nm.

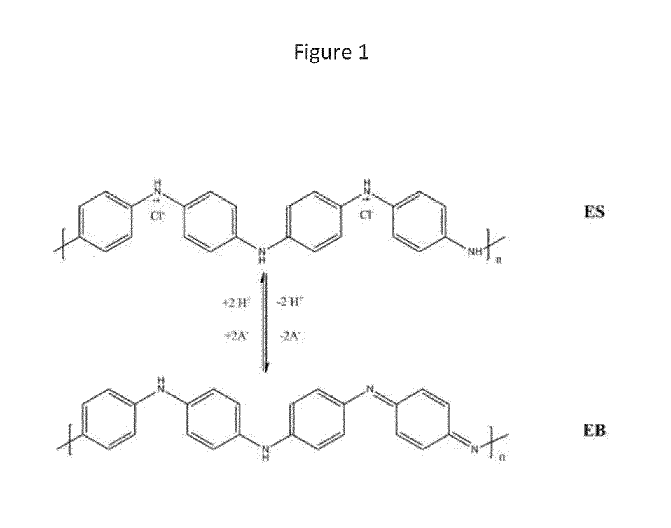

In some cases, the hydrogen selective material 56 includes doped polyaniline. Polyaniline exhibits three different oxidation states: leucoemeraldine (LEB, fully reduced), emeraldine (EB, half-oxidized), and pernigraniline (PNB, fully oxidized). Also, when polyaniline is in the emeraldine state, it can be in either an emeraldine salt or emeraldine base form. When in the emeraldine salt form, the polyaniline is conducting. The emeraldine salt form is usually obtained by protonating the basic amine and imine sites with strong acids. This process is reversible in that the emeraldine base form is obtained by deprotonating the amine groups. Thus, the emeraldine state of polyaniline transitions between an acid form and base form. FIG. 1 shows the acid-base transition of polyaniline.

The acid-base transition of polyaniline renders it pH sensitive and this characteristic allows it to be effectively used in hydrogen detection. When hydrogen contacts an emeraldine salt form of polyaniline, the hydrogen deprotonates the amine groups and converts it to an emeraldine base, which also causes an increase in resistivity and a corresponding decrease in conductivity.

Further, the polyaniline can be doped with a protonic acid to increase its pH sensitivity. A polyaniline with increased pH sensitivity is desirable for hydrogen detection because when hydrogen converts the polyaniline to an emeraldine base, it causes an even larger increase in resistivity and corresponding decrease in conductivity. Larger increases in resistivity (and decreases in conductivity) are desirable because they are easier to detect and increase the sensitivity of the polyaniline to hydrogen.

Polyaniline doped with a protonic acid has an increase in pH sensitivity compared to an undoped polyaniline. In some cases, the polyaniline can be doped with a protonic acid including ions such Cl⁻ and SO₄² to obtain pH sensitivity of around 59 mV. Certain protonic acids cause for an even larger increase in pH sensitivity. For example, polyaniline doped with camphor sulfonic acid has been shown to have a pH sensitivity of around 70 mV.

In some cases, the polyaniline comprises at least one dopant that increases pH sensitivity of the polyaniline. In some cases, the dopant is a protonic acid. In some embodiments, the dopant is hydrochloric acid. In other embodiments, the dopant is camphor sulfonic acid. In yet other embodiments, the dopant is both hydrochloric acid and camphor sulfonic acid. Other possible dopants include sulfuric acid, salicylic acid, acetic acid, citric acid, tartaric acid, oxalic acid, malonic acid, succinic acid, glutamic acid, adipic acid and phthalic acid. Also, in some cases, the polyaniline has a dopant that provides the polyaniline with a pH sensitivity of more than 59 mV. In one embodiment, the polyaniline has a camphor sulfonic acid dopant, which provides the polyaniline with a pH sensitivity of about 70 mV, which is a higher sensitivity observed than when using other dopants.

Also, the hydrogen selective material 56 can be deposited directly onto the conductive material 52 using any desired deposition process. For example, the hydrogen selective material 56 can be deposited onto the conductive material 52 using a spin coating method, a chemical vapor deposition method or a sputtering method. In certain cases, the conductive material 52 is coated with spun cast hydrogen selective material 56. In certain embodiments, the hydrogen selective material 56 is doped polyaniline deposited directly onto the conductive material 52 using a spin coating method. The sensor 50 also includes contact pads 54. The sensor 50 is connected to an electrical circuit via contact pads 54.

The sensor 50 detects hydrogen at very low ppb levels. In some cases, the sensor 50 detects breath hydrogen at levels lower than 40 ppm (<40 ppm) and as high as 100 ppm, thereby covering all hydrogen levels encountered in humans. In certain cases, the sensor 50 detects breath hydrogen at levels lower than 40 ppm. In other cases, the sensor 50 detects breath hydrogen at levels between about 1 ppm and about 100 ppm.

Certain embodiments provide a breath analyzer that detects presence and concentration of hydrogen in a breath sample. The breath analyzer includes an input, a sensor, an electrical circuit and a processor. The input receives a breath sample. The sensor contacts the breath sample. The sensor can have any of the embodiments described herein. In some cases, the sensor includes hydrogen selective material (e.g., doped polyaniline) and a conductive material. The electrical circuit operable connects to the conductive material to the processor. The processor measures resistivity in the electrical circuit and uses the resistivity to calculate a concentration of hydrogen in the breath sample.

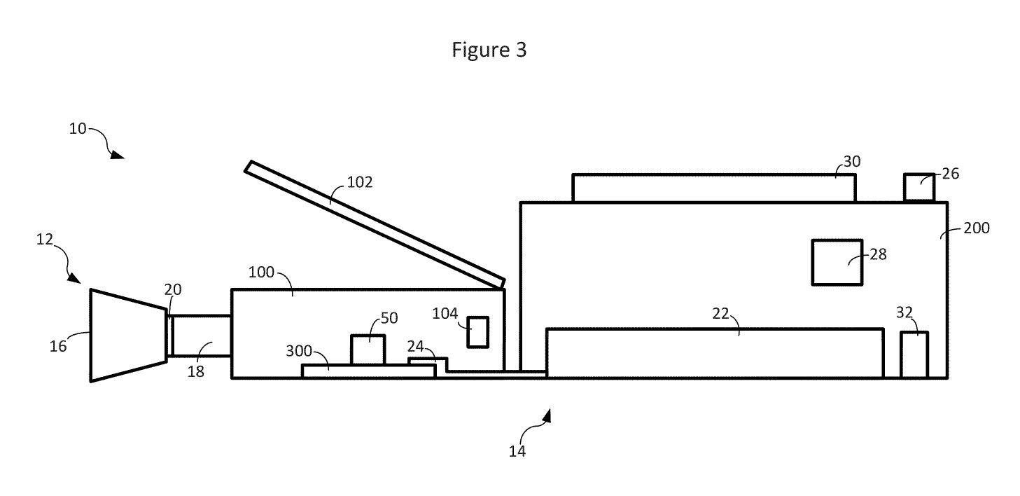

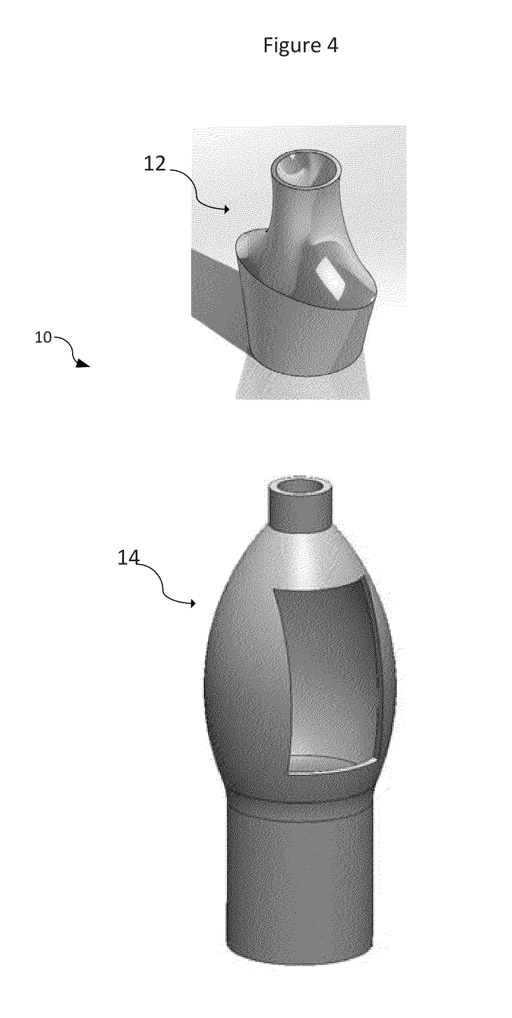

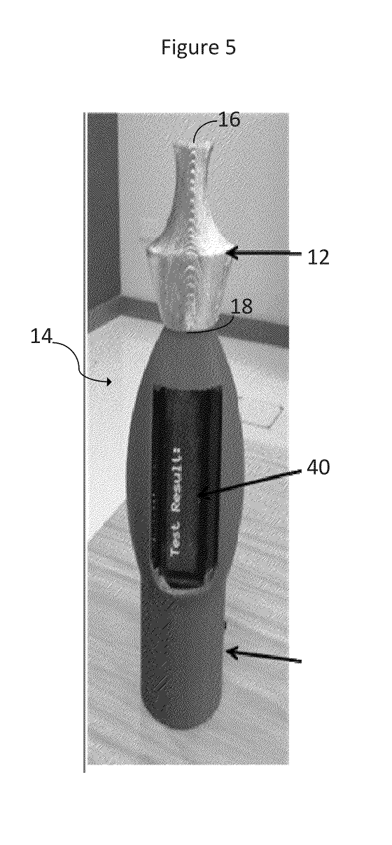

FIG. 3 is a schematic of an exemplary embodiment of a breath analyzer 10. The breath analyzer 10 includes a removable mouthpiece 12 and a main body 14. FIGS. 4-5 also illustrate an exemplary design of the breath analyzer 10. FIG. 4 shows the breath analyzer 10 with a removable mouthpiece 12 and main body 14 in an attached configuration whereas FIG. 5 shows the breath analyzer 10 with the removable mouthpiece 12 attached to the main body 14. The breath analyzer 10 in these embodiments is provided as a self-contained, portable, hand-held device.

The removable mouthpiece 12 can include a first portion 16 and a second portion 18. The first portion 16 can be configured as an input that receives a breath sample. The first portion 16 can also be sized and shaped to receive a user's lips, so that a user can blow exhaled breath into the removable mouthpiece 12. The second portion 18 can be sized and shaped to removably connect to the main body 14. For example, the second portion 18 can be snapped onto or perhaps screwed onto the main body 14.

In some cases, the removable mouthpiece 12 further includes a one-way valve 20. In such cases, a user blows exhaled breath into the mouthpiece 12. The exhaled breath moves forward pass the one-way valve 20 and becomes trapped. In other words, the exhaled breath cannot move backward past the one-way valve 20.

In certain embodiments, the mouthpiece 12 is a single-use mouthpiece. A single-use mouthpiece is desirable because it can be replaced for use with each new user. Also, in some cases, components of the mouthpiece 12 and/or main body 14 in contact with exhaled breath can be made of an inert or non-reactive material (e.g., polytetrafluoroethylene (PFTE)) that does not interfere with hydrogen absorption.

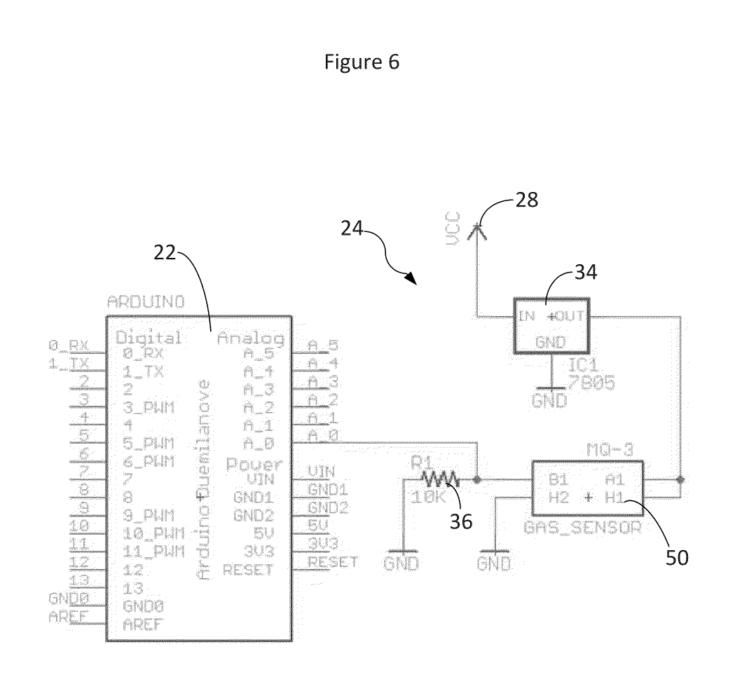

The main body 14 includes a sensor 50, a processor 22 and a power source 28. FIG. 6 is an electrical schematic illustrating the electrical connection between these components according to one embodiment. As shown, the sensor 50, processor 22 and power source 28 are electrically connected via an electrical circuit 24.

The processor 22 can be any desired processor known in the art. In some cases, the processor 22 is a microcontroller. In certain cases, the processor 22 is an Arduino microcontroller.

The sensor can have any of the embodiments already described. In some cases, the sensor includes the sensor 50 of the embodiment of FIG. 2. The sensor 50 is electrically connected to the electrical circuit using any desired connection mechanism. In some cases, the sensor 50 connects to the electrical circuit 24 via an optional sensor mount 300. In such cases, the sensor 50 can be mounted directly onto the sensor mount 300. The sensor mount 300 serves as an interface between the sensor 50 and the electrical circuit 24. Thus, the sensor mount 300 can be any structure known in the art that connects the electrodes 52 of the sensor 50 to the electrical circuit 24. In some embodiments, the sensor mount 300 is a printed circuit board.

In other cases, the sensor 50 is directly connected to the electrical circuit 24. For example, in some embodiments, the electrical circuit 24 includes two metal clips that can be clamped onto the contact pads 54 to create an electrical connection. A user can also replace an old sensor 50 with a new sensor by pulling the old sensor 50 out of the metal clips and inserting a new sensor 50 into the clips.

The main body 14 also includes an on/off button 26 and a power source 28. The power source 28 can be a portable power source, such as a battery. When the on/off button 26 is activated, the power source 28 turns on. As shown in FIG. 6, the power source 28 supplies voltage to a voltage regulator 24. In certain cases, the power source 28 supplies 9 volts to the voltage regulator 24. The voltage regulator 24 regulates the amount of voltage sent to the sensor 50. In some cases, the voltage regulator 24 supplies a voltage to the sensor 50 in the amount of between 0 volts to 5 volts. In certain cases, the voltage regulator 24 supplies a voltage to the sensor 50 in the amount of about 5 volts. In one embodiment, the voltage regulator 24 is an IC1 7805 voltage regulator, a product manufactured by Fairchild Electronics.

A resistor 36 is also electrically connected to the sensor 50 and provides resistance to the sensor. In some case, the resister 36 is a 10 kΩ resistor. When the breath sample contacts the sensor 50, a change in resistivity occurs in the sensor 50 that correlates to an amount of hydrogen in the sample. The sensor 50 outputs voltage (along with the changes in resistivity) to the processor 22. The processor 22 detects changes in resistivity in and uses the changes in resistivity to calculate a concentration of hydrogen in the breath sample. The processor 22 can also compare a concentration of hydrogen between two different breath samples.

The main body 14 can also include a display 30 operably connected to the processor 22. In some cases, the display 30 shows the concentration of hydrogen calculated by the processor 22. In other cases, the display 30 shows results of a comparison between concentrations of hydrogen between two or more different breath samples. In some cases, the comparison results can show a positive comparison result or a negative comparison result that is determined using a predetermined positive/negative threshold. In other cases, the comparison results can show a numerical value that is the delta between a baseline hydrogen value and a post-substrate ingestion hydrogen value. The main body 14 can also include an optional wireless connector 32 (e.g., a Bluetooth connector) that transmits data calculated by the processor 22 to an external computer.

In some embodiments, the main body 14 is configured as including a first compartment 100 and a second compartment 200. In some cases, as shown in FIG. 3, the first compartment 100 is a chamber that houses a sensor 50 and sensor mount 300 and the second compartment 200 is an electrical housing that houses various components.

In some cases, the chamber 100 includes a lid or door 102 that opens and shuts. When the door 102 is closed, the chamber 100 provides a closed, sealed environment around the sensor 50. When the door 102 is open, the sensor 50 is accessible through the door opening. A user can open the chamber door 102 to remove and replace the sensor 50 as needed. The chamber 100 also includes an outlet 104. The outlet 104 includes a cap that can be opened to release a breath sample from the chamber 100 and closed to trap a breath sample within the chamber 100.

The removable mouthpiece 12 is connected to the chamber 100 such that exhaled breath passes from the mouthpiece 12 directly into the chamber 100. The breath analyzer 10 can include an optional desiccant assembly 60. The desiccant assembly 60 helps to remove excess moisture from the exhaled breath. In some embodiments, the desiccant assembly 60 is provided inside of the mouthpiece 12. In other embodiments, the desiccant assembly 60 is provided inside of the chamber 100. Exhaled breath first moves through the desiccant assembly 60 before coming into contact with the sensor 50.

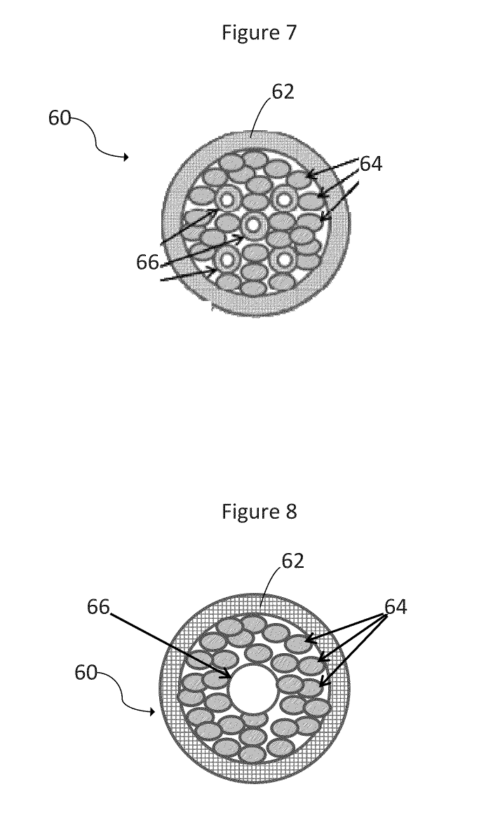

FIG. 7 shows an exemplary embodiment of a desiccant assembly 60. In some cases, the desiccant assembly is provided as a tube 62 upon which exhaled air flows through. The tube 62 can have an interior filled with a plurality of desiccant beads 64. The desiccant beads 64 can also be arranged such that a plurality of channels 66 are created for exhaled air to flow through. FIG. 8 shows another exemplary embodiment of a desiccant assembly 60. In this embodiment, the tube can have an interior filled with a plurality of desiccant beads 64 arranged such that a single channel 66 is created.

Still further, the breath analyzer 10 can include an optional gas filter (not shown). The gas filter helps to remove a selected gas (e.g., carbon dioxide, nitrogen, ammonia and/or hydrogen) from the exhaled breath. In some embodiments, the gas filter is provided inside of the mouthpiece 12. In other embodiments, the gas filter is provided inside of the chamber 100. Exhaled breath first moves through the gas filter before coming into contact with the sensor 50.

Referring back to FIG. 3, the main body 14 can also include a second compartment configured as an electrical housing 200 that houses various components. In the illustrate embodiment, the electrical housing 200 houses the processor 22, parts of the electrical circuit 24, a power source 28, a display 30, and a wireless connector 32.

During use, a user turns the breath analyzer 10 on by activating the on/off switch 26. The on/off switch 26 can be located anywhere about an exterior surface of the main body 14. This on/off switch 26 in turn prompts the power source 28 to supply voltage to the voltage regulator 34. The voltage regulator 34 regulates and supplies voltage to the sensor 50. The resistor 36 also supplies resistance to the sensor 50.

A user then blows a breath sample into the first portion 16 of the mouthpiece 12. The breath sample moves pass the one way valve 20 and through the optional desiccant/gas filter 60. The breath sample then moves out of the optional desiccant/gas filter 60 and into the chamber 100 where it contacts the sensor 50. The breath sample causes a change in resistivity to occur in the sensor 50. This resistivity is outputted to the processor 22.

Other embodiments provide a breath test method for screening for a gastrointestinal disorder. Some embodiments provide a breath test method for screening for celiac disease. In some cases, the screening method includes steps of: collecting a fasting breath sample from a subject, exposing the fasting breath sample to a sensor comprising hydrogen selective material having a resistivity that increases in response to increased presence of hydrogen, measuring resistivity the sensor that occurs when the fasting breath sample contacts the sensor and designating the subject as having an increased likelihood of having a celiac disease if the measured resistivity is above and/or beneath a predetermined value. The fasting breath sample can include a single sample from a subject after fasting. In some cases, the fasting breath sample is a sample from a subject after fasting for at least 2 hours, 3 hours, 4 hours, 5 hours, 6 hours, 7 hours or 8 hours.

In some cases, the breath test screening method for celiac disease is performed in connection with a breath analyzer. The breath analyzer can have any of the embodiments described herein. In some cases, the breath analyzer includes a portable, hand-held breath analyzer. The breath test screening method includes steps of: providing a breath analyzer having a sensor comprising hydrogen selective material having a resistivity that increases in response to increased presence of hydrogen, prompting a subject to exhale a fasting breath sample into the breath analyzer, allowing the breath analyzer to measure a resistivity of the sensor, and designating the subject as having an increased likelihood of having celiac disease if the measured resistivity is above and/or beneath a predetermined value.

In some cases, the breath analyzer includes a main body and a removable mouthpiece, wherein the removable mouthpiece removably attaches to the main body, wherein the main body includes a sensor, a processor, a power source and an electrical circuit, wherein the electrical circuit operably connects the power source to the sensor and connects the sensor to the processor, wherein the sensor comprises a conductive material and hydrogen selective material in contact with the conductive material, wherein the hydrogen selective material has a resistivity that increases in response to increased concentration of hydrogen. The breath test screening method includes steps of: turning on a breath analyzer by activating an on/off switch, prompting a subject to exhale a fasting breath sample into the removable mouthpiece, allowing the processor to measure a resistivity of the sensor that occurs when the fasting breath sample contacts the sensor, and designating the subject as having an increased likelihood of having celiac disease if the measured resistivity is above and/or beneath a predetermined value.

Other embodiments provide a breath test method for diagnosing a subject with celiac disease. The breath test diagnostic method includes steps of: collecting a fasting breath sample from a subject, exposing the fasting breath sample to a sensor comprising hydrogen selective material having a resistivity that increases in response to increased presence of hydrogen, measuring resistivity the fasting breath sample and diagnosing the subject with celiac disease if the measured resistivity is above and/or beneath a predetermined value.

In some cases, the breath test diagnostic method for celiac disease is performed in connection with a breath analyzer. In some cases, the breath analyzer includes a portable, hand-held breath analyzer. The breath test diagnostic method includes steps of: providing a breath analyzer having a sensor comprising hydrogen selective material having a resistivity that increases in response to increased presence of hydrogen, prompting a subject to exhale a fasting breath sample into the breath analyzer, allowing the breath analyzer to measure a resistivity of the sensor, and diagnosing the subject with celiac disease if the measured resistivity is above and/or beneath a predetermined value.

In some cases, the breath analyzer includes a main body and a removable mouthpiece, wherein the removable mouthpiece removably attaches to the main body, wherein the main body includes a sensor, a processor, a power source and an electrical circuit, wherein the electrical circuit operably connects the power source to the sensor and connects the sensor to the processor, wherein the sensor comprises a conductive material and hydrogen selective material in contact with the conductive material, wherein the hydrogen selective material has a resistivity that increases in response to increased concentration of hydrogen. The breath test screening method includes steps of: turning on a breath analyzer by activating an on/off switch, prompting the subject to exhale a fasting breath sample into the removable mouthpiece, allowing the processor to measure a resistivity of the sensor that occurs when the fasting breath sample contacts the sensor, and diagnosing the subject with celiac disease if the measured resistivity is above and/or beneath a predetermined value.

Some embodiments provide a breath test method for screening for NCGS. In some cases, the screening method includes steps of: collecting a fasting breath sample from a subject, exposing the fasting breath sample to a sensor comprising hydrogen selective material having a resistivity that increases in response to increased presence of hydrogen, measuring resistivity the sensor that occurs when the fasting breath sample contacts the sensor and designating the subject as having an increased likelihood of having a NCGS if the measured resistivity is above and/or beneath a predetermined value. The fasting breath sample can include a single sample from a subject after fasting. In some cases, the fasting breath sample is a sample from a subject after fasting for at least 2 hours, 3 hours, 4 hours, 5 hours, 6 hours, 7 hours or 8 hours.

In some cases, the breath test screening method for 3 is performed in connection with a breath analyzer. The breath analyzer can have any of the embodiments described herein. In some cases, the breath analyzer includes a portable, hand-held breath analyzer. The breath test screening method includes steps of: providing a breath analyzer having a sensor comprising hydrogen selective material having a resistivity that increases in response to increased presence of hydrogen, prompting a subject to exhale a fasting breath sample into the breath analyzer, allowing the breath analyzer to measure a resistivity of the sensor, and designating the subject as having an increased likelihood of having NCGS if the measured resistivity is above and/or beneath a predetermined value.

In some cases, the breath analyzer includes a main body and a removable mouthpiece, wherein the removable mouthpiece removably attaches to the main body, wherein the main body includes a sensor, a processor, a power source and an electrical circuit, wherein the electrical circuit operably connects the power source to the sensor and connects the sensor to the processor, wherein the sensor comprises a conductive material and hydrogen selective material in contact with the conductive material, wherein the hydrogen selective material has a resistivity that increases in response to increased concentration of hydrogen. The breath test screening method includes steps of: turning on a breath analyzer by activating an on/off switch, prompting a subject to exhale a fasting breath sample into the removable mouthpiece, allowing the processor to measure a resistivity of the sensor that occurs when the fasting breath sample contacts the sensor, and designating the subject as having an increased likelihood of having NCGS if the measured resistivity is above and/or beneath a predetermined value.

Other embodiments provide a breath test method for diagnosing a subject with NCGS. The breath test diagnostic method includes steps of: collecting a fasting breath sample from a subject, exposing the fasting breath sample to a sensor comprising hydrogen selective material having a resistivity that increases in response to increased presence of hydrogen, measuring resistivity the fasting breath sample and diagnosing the subject with NCGS if the measured resistivity is above and/or beneath a predetermined value.

In some cases, the breath test diagnostic method for NCGS is performed in connection with a breath analyzer. In some cases, the breath analyzer includes a portable, hand-held breath analyzer. The breath test diagnostic method includes steps of: providing a breath analyzer having a sensor comprising hydrogen selective material having a resistivity that increases in response to increased presence of hydrogen, prompting a subject to exhale a fasting breath sample into the breath analyzer, allowing the breath analyzer to measure a resistivity of the sensor, and diagnosing the subject with NCGS if the measured resistivity is above and/or beneath a predetermined value.

In some cases, the breath analyzer includes a main body and a removable mouthpiece, wherein the removable mouthpiece removably attaches to the main body, wherein the main body includes a sensor, a processor, a power source and an electrical circuit, wherein the electrical circuit operably connects the power source to the sensor and connects the sensor to the processor, wherein the sensor comprises a conductive material and hydrogen selective material in contact with the conductive material, wherein the hydrogen selective material has a resistivity that increases in response to increased concentration of hydrogen. The breath test screening method includes steps of: turning on a breath analyzer by activating an on/off switch, prompting the subject to exhale a fasting breath sample into the removable mouthpiece, allowing the processor to measure a resistivity of the sensor that occurs when the fasting breath sample contacts the sensor, and diagnosing the subject with NCGS if the measured resistivity is above and/or beneath a predetermined value.

Other embodiments provide a breath test method for screening for lactose intolerance. The breath test screening method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a post-lactose breath sample from a subject, exposing the post-lactose breath sample to a sensor comprising hydrogen selective material having a resistivity that increases in response to increased presence of hydrogen, measuring resistivity of the post-lactose breath sample, comparing the measured resistivity of the baseline breath sample and the post-lactose breath sample and designating the subject has having an increased likelihood of having lactose intolerance when comparing meets a predetermined criteria. The baseline breath sample can obtained after fasting for a time period and before ingesting the lactose-containing material. The post-lactose breath sample can include a sample from the same subject after ingesting a lactose-containing meal. The post-lactose breath sample can be obtained at a time period after ingesting the lactose-containing material. For example, the post-lactose breath sample can obtained at a time period of 20-30 minutes after ingesting the lactose-containing material. In some cases, the post-lactose breath sample is obtained at a time period of 30 minutes after ingesting the lactose-containing material.

In some embodiments, the step of collecting a post-lactose breath sample comprises collecting post-lactose breath samples at a plurality different time points. For example, in some cases, the plurality of time points include at least a first time point and a second time point. In other cases, the plurality of time points include at least a first time point, a second time point, a third time point and a fourth time point. In other cases, the plurality of time points include at least a first time point, a second time point, a third time point, a fourth time point and a fifth time point. In yet other cases, the plurality of time points include at least a first time point, a second time point, a third time point, a fourth time point, a fifth time point and a sixth time point. The time points can be spaced at any desired interval, such as a 15 minute interval, a 20 minute interval or a 30 minute interval. In certain cases, the first time point is a 15 minute time point, the second time point is a 30 minute time point, the third time point is a 45 minute time point, the fourth time point is a 60 minute time point, the fifth time point is a 75 minute time point and the sixth time point is a 90 minute time point. In other cases, the first time point is a 30 minute time point, the second time point is a 60 minute time point, the third time point is a 90 minute time point, the fourth time point is a 120 minute time point, the fifth time point is a 150 minute time point and the sixth time point is a 180 minute time point. Likewise, any desired number of time points can be used and the time points can be spaced by any desired time interval.

In some cases, the screening method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a first post-lactose breath sample from a subject at a first time point, exposing the first post-lactose breath sample to a sensor comprising hydrogen selective material having a resistivity that increases in response to increased presence of hydrogen, measuring resistivity of the first post-lactose breath sample, collecting a second post-lactose breath sample from a subject at a second time point, exposing the second post-lactose breath sample to a sensor comprising hydrogen selective material having a resistivity that increases in response to increased presence of hydrogen, measuring resistivity of the second post-lactose breath sample, comparing the resistivity of the baseline breath sample, first post-lactose breath sample and second post-lactose breath sample and designating the subject has having an increased likelihood of having lactose intolerance when the comparing meets a predetermined criteria.

In some cases, the step of collecting a post-lactose breath sample comprises collecting a plurality of post-lactose breath samples at four time points. In such cases, the screening method includes steps of: collecting a baseline breath sample from a subject, exposing the baseline breath sample to a sensor comprising hydrogen selective material having a resistivity that increases in response to increased presence of hydrogen, measuring a resistivity of the sensor that occurs when the baseline breath sample contacts the sensor, prompting the subject to ingest a lactose-containing meal, collecting a first post-lactose breath sample from a subject at a first time point, exposing the first post-lactose breath sample to the sensor, measuring a resistivity of the sensor that occurs when the first post-lactose breath sample contacts the sensor, collecting a second post-lactose breath sample from a subject at a second time point, exposing the second post-lactose breath sample to the sensor, measuring a resistivity of the sensor that occurs when the second post-lactose breath sample contacts the sensor, collecting a third post-lactose breath sample from a subject at a third time point, exposing the third post-lactose breath sample to the sensor, measuring a resistivity of the sensor that occurs when the third post-lactose breath sample contacts the sensor, collecting a fourth post-lactose breath sample from a subject at a second time point, exposing the fourth post-lactose breath sample to the sensor, measuring a resistivity of the sensor that occurs when the fourth post-lactose breath sample contacts the sensor, comparing the resistivity of the baseline breath sample, the first post-lactose breath sample, the second post-lactose breath sample, the third post-lactose breath sample and the fourth post-lactose breath sample and designating the subject has having an increased likelihood of having lactose intolerance when the comparing meets a predetermined criteria.

In some cases, the breath test screening method for screening for lactose intolerance is performed in connection with a breath analyzer. The breath analyzer can have any of the embodiments described herein. In some cases, the breath analyzer includes a portable, hand-held breath analyzer. The breath test diagnostic method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs when the baseline breath sample contacts the sensor, resetting the breath analyzer, prompting a subject to exhale a post-lactose breath sample into the breath analyzer, allowing the breath analyzer to measure a resistivity of the sensor that occurs when the post-lactose ingestion breath sample contacts the sensor, comparing the measured resistivity of the baseline breath sample to the measured resistivity of the post-lactose breath sample and designating the subject as having an increased likelihood of having lactose intolerance when the comparing meets a predetermined criteria.

In other cases, the breath test screening method includes steps of: providing a breath analyzer having a sensor comprising hydrogen selective material having a resistivity that increases in response to increased presence of hydrogen, prompting a subject to exhale a baseline breath sample into the breath analyzer for a time period, allowing the breath analyzer to measure a resistivity of the sensor that occurs with the baseline breath sample, resetting the breath analyzer, prompting a subject to exhale a first post-lactose breath sample into the breath analyzer, allowing the breath analyzer to measure a resistivity of the sensor that occurs with the first post-lactose ingestion breath sample, resetting the breath analyzer, prompting a subject to exhale a second post-lactose breath sample into the breath analyzer, allowing the breath analyzer to measure a resistivity of the sensor that occurs with the second post-lactose ingestion breath sample, comparing the measured resistivity of the baseline breath sample, the first post-lactose breath sample and the second post-lactose breath sample, and designating the subject as having an increased likelihood of having lactose intolerance when the comparing meets a predetermined criteria.

In other cases, the breath test method for screening for lactose intolerance includes the steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-lactose breath sample into the breath analyzer, allowing the breath analyzer to measure a resistivity of the sensor that occurs with the first post-lactose ingestion breath sample, resetting the breath analyzer, prompting a subject to exhale a second post-lactose breath sample into the breath analyzer, allowing the breath analyzer to measure a resistivity of the sensor that occurs with the second post-lactose ingestion breath sample, resetting the breath analyzer, prompting a subject to exhale a third post-lactose breath sample into the breath analyzer, allowing the breath analyzer to measure a resistivity of the sensor that occurs with the third post-lactose ingestion breath sample, resetting the breath analyzer, prompting a subject to exhale a fourth post-lactose breath sample into the breath analyzer, allowing the breath analyzer to measure a resistivity of the sensor that occurs with the fourth post-lactose ingestion breath sample, comparing the measured resistivity of the baseline breath sample, the first post-lactose breath sample, the second post-lactose breath sample, the third post-lactose breath sample and the fourth post-lactose breath sample, and designating the subject as having an increased likelihood of having lactose intolerance when the comparing meets a predetermined criteria.

Other embodiments provide a breath test method for diagnosing lactose intolerance. The breath test screening method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a post-lactose breath sample from a subject, exposing the post-lactose breath sample to a sensor comprising hydrogen selective material having a resistivity that increases in response to increased presence of hydrogen, measuring resistivity of the post-lactose breath sample, comparing the measured resistivity of the baseline breath sample and the post-lactose breath sample and diagnosing the subject with lactose intolerance when the comparing meets a predetermined criteria.

In some cases, the step of collecting a post-lactose breath sample comprises collecting a plurality of post-lactose breath samples at different time points. In such cases, the diagnostic method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a first post-lactose breath sample from a subject at a first time point, exposing the first post-lactose breath sample to a sensor comprising hydrogen selective material having a resistivity that increases in response to increased presence of hydrogen, measuring resistivity of the first post-lactose breath sample, collecting a second post-lactose breath sample from a subject at a second time point, exposing the second post-lactose breath sample to a sensor comprising hydrogen selective material having a resistivity that increases in response to increased presence of hydrogen, measuring resistivity of the second post-lactose breath sample, comparing the resistivity of the baseline breath sample, first post-lactose breath sample and second post-lactose breath sample and diagnosing the subject with lactose intolerance when the comparing meets a predetermined criteria.

In some cases, the step of collecting a post-lactose breath sample comprises collecting a plurality of post-lactose breath samples at four time points. In such cases, the screening method includes steps of: collecting a baseline breath sample from a subject, exposing the baseline breath sample to a sensor comprising hydrogen selective material having a resistivity that increases in response to increased presence of hydrogen, measuring a resistivity of the sensor that occurs when the baseline breath sample contacts the sensor, prompting the subject to ingest a lactose-containing meal, collecting a first post-lactose breath sample from a subject at a first time point, exposing the first post-lactose breath sample to the sensor, measuring a resistivity of the sensor that occurs when the first post-lactose breath sample contacts the sensor, collecting a second post-lactose breath sample from a subject at a second time point, exposing the second post-lactose breath sample to the sensor, measuring a resistivity of the sensor that occurs when the second post-lactose breath sample contacts the sensor, collecting a third post-lactose breath sample from a subject at a third time point, exposing the third post-lactose breath sample to the sensor, measuring a resistivity of the sensor that occurs when the third post-lactose breath sample contacts the sensor, collecting a fourth post-lactose breath sample from a subject at a second time point, exposing the fourth post-lactose breath sample to the sensor, measuring a resistivity of the sensor that occurs when the fourth post-lactose breath sample contacts the sensor, comparing the resistivity of the baseline breath sample, the first post-lactose breath sample, the second post-lactose breath sample, the third post-lactose breath sample and the fourth post-lactose breath sample and diagnosing the subject with lactose intolerance when the comparing meets a predetermined criteria.

In some cases, the breath test diagnostic method is performed in connection with a breath analyzer. The breath analyzer can have any of the embodiments described herein. In some cases, the breath analyzer includes a portable, hand-held breath analyzer. The breath test diagnostic method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs when the baseline breath sample contacts the sensor, resetting the breath analyzer, prompting a subject to exhale a post-lactose breath sample into the breath analyzer, allowing the breath analyzer to measure a resistivity of the sensor that occurs when the post-lactose ingestion breath sample contacts the sensor, comparing the measured resistivity of the baseline breath sample to the measured resistivity of the post-lactose breath sample and diagnosing the subject with lactose intolerance when the comparing meets a predetermined criteria.

In other cases, the breath test diagnostic method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-lactose breath sample into the breath analyzer, allowing the breath analyzer to measure a resistivity of the sensor that occurs with the first post-lactose ingestion breath sample, resetting the breath analyzer, prompting a subject to exhale a second post-lactose breath sample into the breath analyzer, allowing the breath analyzer to measure a resistivity of the sensor that occurs with the second post-lactose ingestion breath sample, comparing the measured resistivity of the baseline breath sample, the first post-lactose breath sample and the second post-lactose breath sample, and diagnosing the subject with lactose intolerance when the comparing meets a predetermined criteria.

In other cases, the breath test diagnostic method includes the steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-lactose breath sample into the breath analyzer, allowing the breath analyzer to measure a resistivity of the sensor that occurs with the first post-lactose ingestion breath sample, resetting the breath analyzer, prompting a subject to exhale a second post-lactose breath sample into the breath analyzer, allowing the breath analyzer to measure a resistivity of the sensor that occurs with the second post-lactose ingestion breath sample, resetting the breath analyzer, prompting a subject to exhale a third post-lactose breath sample into the breath analyzer, allowing the breath analyzer to measure a resistivity of the sensor that occurs with the third post-lactose ingestion breath sample, resetting the breath analyzer, prompting a subject to exhale a fourth post-lactose breath sample into the breath analyzer, allowing the breath analyzer to measure a resistivity of the sensor that occurs with the fourth post-lactose ingestion breath sample, comparing the measured resistivity of the baseline breath sample, the first post-lactose breath sample, the second post-lactose breath sample, the third post-lactose breath sample and the fourth post-lactose breath sample, and diagnosing the subject with lactose intolerance when the comparing meets a predetermined criteria.

Other embodiments provide a breath test method for screening for fructose intolerance. The breath test screening method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a post-fructose breath sample from a subject, exposing the post-fructose breath sample to a sensor comprising hydrogen selective material having a resistivity that increases in response to increased presence of hydrogen, measuring resistivity of the post-fructose breath sample, comparing the measured resistivity of the baseline breath sample and the post-fructose breath sample and designating the subject has having an increased likelihood of having fructose intolerance when comparing meets a predetermined criteria. The baseline breath sample can obtained after fasting for a time period and before ingesting the fructose-containing material. The post-fructose breath sample can include a sample from the same subject after ingesting a fructose-containing meal. The post-fructose breath sample can be obtained at a time period after ingesting the fructose-containing material. In some cases, the post-fructose breath sample is obtained at a time period of 20-30 minutes after ingesting the fructose-containing material. In other cases, the post-fructose breath sample is obtained at a time period of 30 minutes after ingesting the fructose-containing material.

In some embodiments, the step of collecting a post-fructose breath sample comprises collecting post-fructose breath samples at a plurality different time points. For example, in some cases, the plurality of time points include at least a first time point and a second time point. In other cases, the plurality of time points include at least a first time point, a second time point, a third time point and a fourth time point. In other cases, the plurality of time points include at least a first time point, a second time point, a third time point, a fourth time point and a fifth time point. In yet other cases, the plurality of time points include at least a first time point, a second time point, a third time point, a fourth time point, a fifth time point and a sixth time point. The time points can be spaced at any desired interval, such as a 15 minute interval, a 20 minute interval or a 30 minute interval. In certain cases, the first time point is a 15 minute time point, the second time point is a 30 minute time point, the third time point is a 45 minute time point, the fourth time point is a 60 minute time point, the fifth time point is a 75 minute time point and the sixth time point is a 90 minute time point. In other cases, the first time point is a 30 minute time point, the second time point is a 60 minute time point, the third time point is a 90 minute time point, the fourth time point is a 120 minute time point, the fifth time point is a 150 minute time point and the sixth time point is a 180 minute time point. Likewise, any desired number of time points can be used and the time points can be spaced by any desired time interval.

In some cases, the screening method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a first post-fructose breath sample from a subject at a first time point, exposing the first post-fructose breath sample to a sensor comprising hydrogen selective material having a resistivity that increases in response to increased presence of hydrogen, measuring resistivity of the first post-fructose breath sample, collecting a second post-fructose breath sample from a subject at a second time point, exposing the second post-fructose breath sample to a sensor comprising hydrogen selective material having a resistivity that increases in response to increased presence of hydrogen, measuring resistivity of the second post-fructose breath sample, comparing the resistivity of the baseline breath sample, first post-fructose breath sample and second post-fructose breath sample and designating the subject has having an increased likelihood of having fructose intolerance when the comparing meets a predetermined criteria.

In some cases, the step of collecting a post-fructose breath sample comprises collecting a plurality of post-fructose breath samples at four time points. In such cases, the screening method includes steps of: collecting a baseline breath sample from a subject, exposing the baseline breath sample to a sensor comprising hydrogen selective material having a resistivity that increases in response to increased presence of hydrogen, measuring a resistivity of the sensor that occurs when the baseline breath sample contacts the sensor, prompting the subject to ingest a fructose-containing meal, collecting a first post-fructose breath sample from a subject at a first time point, exposing the first post-fructose breath sample to the sensor, measuring a resistivity of the sensor that occurs when the first post-fructose breath sample contacts the sensor, collecting a second post-fructose breath sample from a subject at a second time point, exposing the second post-fructose breath sample to the sensor, measuring a resistivity of the sensor that occurs when the second post-fructose breath sample contacts the sensor, collecting a third post-fructose breath sample from a subject at a third time point, exposing the third post-fructose breath sample to the sensor, measuring a resistivity of the sensor that occurs when the third post-fructose breath sample contacts the sensor, collecting a fourth post-fructose breath sample from a subject at a second time point, exposing the fourth post-fructose breath sample to the sensor, measuring a resistivity of the sensor that occurs when the fourth post-fructose breath sample contacts the sensor, comparing the resistivity of the baseline breath sample, the first post-fructose breath sample, the second post-fructose breath sample, the third post-fructose breath sample and the fourth post-fructose breath sample and designating the subject has having an increased likelihood of having fructose intolerance when the comparing meets a predetermined criteria.

In some cases, the breath test screening method for screening for fructose intolerance is performed in connection with a breath analyzer. The breath analyzer can have any of the embodiments described herein. In some cases, the breath analyzer includes a portable, hand-held breath analyzer. The breath test diagnostic method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs when the baseline breath sample contacts the sensor, resetting the breath analyzer, prompting a subject to exhale a post-fructose breath sample into the breath analyzer, allowing the breath analyzer to measure a resistivity of the sensor that occurs when the post-fructose ingestion breath sample contacts the sensor, comparing the measured resistivity of the baseline breath sample to the measured resistivity of the post-fructose breath sample and designating the subject as having an increased likelihood of having fructose intolerance when the comparing meets a predetermined criteria.

In other cases, the breath test screening method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-fructose breath sample into the breath analyzer, allowing the breath analyzer to measure a resistivity of the sensor that occurs with the first post-fructose ingestion breath sample, resetting the breath analyzer, prompting a subject to exhale a second post-fructose breath sample into the breath analyzer, allowing the breath analyzer to measure a resistivity of the sensor that occurs with the second post-fructose ingestion breath sample, comparing the measured resistivity of the baseline breath sample, the first post-fructose breath sample and the second post-fructose breath sample, and designating the subject as having an increased likelihood of having fructose intolerance when the comparing meets a predetermined criteria.

In other cases, the breath test method for screening for fructose intolerance includes the steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-fructose breath sample into the breath analyzer, allowing the breath analyzer to measure a resistivity of the sensor that occurs with the first post-fructose ingestion breath sample, resetting the breath analyzer, prompting a subject to exhale a second post-fructose breath sample into the breath analyzer, allowing the breath analyzer to measure a resistivity of the sensor that occurs with the second post-fructose ingestion breath sample, resetting the breath analyzer, prompting a subject to exhale a third post-fructose breath sample into the breath analyzer, allowing the breath analyzer to measure a resistivity of the sensor that occurs with the third post-fructose ingestion breath sample, resetting the breath analyzer, prompting a subject to exhale a fourth post-fructose breath sample into the breath analyzer, allowing the breath analyzer to measure a resistivity of the sensor that occurs with the fourth post-fructose ingestion breath sample, comparing the measured resistivity of the baseline breath sample, the first post-fructose breath sample, the second post-fructose breath sample, the third post-fructose breath sample and the fourth post-fructose breath sample, and designating the subject as having an increased likelihood of having fructose intolerance when the comparing meets a predetermined criteria.

Other embodiments provide a breath test method for diagnosing fructose intolerance. The breath test screening method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a post-fructose breath sample from a subject, exposing the post-fructose breath sample to a sensor comprising hydrogen selective material having a resistivity that increases in response to increased presence of hydrogen, measuring resistivity of the post-fructose breath sample, comparing the measured resistivity of the baseline breath sample and the post-fructose breath sample and diagnosing the subject with fructose intolerance when the comparing meets a predetermined criteria.

In some cases, the step of collecting a post-fructose breath sample comprises collecting a plurality of post-fructose breath samples at different time points. In such cases, the diagnostic method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a first post-fructose breath sample from a subject at a first time point, exposing the first post-fructose breath sample to a sensor comprising hydrogen selective material having a resistivity that increases in response to increased presence of hydrogen, measuring resistivity of the first post-fructose breath sample, collecting a second post-fructose breath sample from a subject at a second time point, exposing the second post-fructose breath sample to a sensor comprising hydrogen selective material having a resistivity that increases in response to increased presence of hydrogen, measuring resistivity of the second post-fructose breath sample, comparing the resistivity of the baseline breath sample, first post-fructose breath sample and second post-fructose breath sample and diagnosing the subject with fructose intolerance when the comparing meets a predetermined criteria.

In some cases, the step of collecting a post-fructose breath sample comprises collecting a plurality of post-fructose breath samples at four time points. In such cases, the screening method includes steps of: collecting a baseline breath sample from a subject, exposing the baseline breath sample to a sensor comprising hydrogen selective material having a resistivity that increases in response to increased presence of hydrogen, measuring a resistivity of the sensor that occurs when the baseline breath sample contacts the sensor, prompting the subject to ingest a fructose-containing meal, collecting a first post-fructose breath sample from a subject at a first time point, exposing the first post-fructose breath sample to the sensor, measuring a resistivity of the sensor that occurs when the first post-fructose breath sample contacts the sensor, collecting a second post-fructose breath sample from a subject at a second time point, exposing the second post-fructose breath sample to the sensor, measuring a resistivity of the sensor that occurs when the second post-fructose breath sample contacts the sensor, collecting a third post-fructose breath sample from a subject at a third time point, exposing the third post-fructose breath sample to the sensor, measuring a resistivity of the sensor that occurs when the third post-fructose breath sample contacts the sensor, collecting a fourth post-fructose breath sample from a subject at a second time point, exposing the fourth post-fructose breath sample to the sensor, measuring a resistivity of the sensor that occurs when the fourth post-fructose breath sample contacts the sensor, comparing the resistivity of the baseline breath sample, the first post-fructose breath sample, the second post-fructose breath sample, the third post-fructose breath sample and the fourth post-fructose breath sample and diagnosing the subject with fructose intolerance when the comparing meets a predetermined criteria.

In some cases, the breath test diagnostic method is performed in connection with a breath analyzer. The breath analyzer can have any of the embodiments described herein. In some cases, the breath analyzer includes a portable, hand-held breath analyzer. The breath test diagnostic method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs when the baseline breath sample contacts the sensor, resetting the breath analyzer, prompting a subject to exhale a post-fructose breath sample into the breath analyzer, allowing the breath analyzer to measure a resistivity of the sensor that occurs when the post-fructose ingestion breath sample contacts the sensor, comparing the measured resistivity of the baseline breath sample to the measured resistivity of the post-fructose breath sample and diagnosing the subject with fructose intolerance when the comparing meets a predetermined criteria.

In other cases, the breath test diagnostic method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-fructose breath sample into the breath analyzer, allowing the breath analyzer to measure a resistivity of the sensor that occurs with the first post-fructose ingestion breath sample, resetting the breath analyzer, prompting a subject to exhale a second post-fructose breath sample into the breath analyzer, allowing the breath analyzer to measure a resistivity of the sensor that occurs with the second post-fructose ingestion breath sample, comparing the measured resistivity of the baseline breath sample, the first post-fructose breath sample and the second post-fructose breath sample, and diagnosing the subject with fructose intolerance when the comparing meets a predetermined criteria.

In other cases, the breath test diagnostic method includes the steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-fructose breath sample into the breath analyzer, allowing the breath analyzer to measure a resistivity of the sensor that occurs with the first post-fructose ingestion breath sample, resetting the breath analyzer, prompting a subject to exhale a second post-fructose breath sample into the breath analyzer, allowing the breath analyzer to measure a resistivity of the sensor that occurs with the second post-fructose ingestion breath sample, resetting the breath analyzer, prompting a subject to exhale a third post-fructose breath sample into the breath analyzer, allowing the breath analyzer to measure a resistivity of the sensor that occurs with the third post-fructose ingestion breath sample, resetting the breath analyzer, prompting a subject to exhale a fourth post-fructose breath sample into the breath analyzer, allowing the breath analyzer to measure a resistivity of the sensor that occurs with the fourth post-fructose ingestion breath sample, comparing the measured resistivity of the baseline breath sample, the first post-fructose breath sample, the second post-fructose breath sample, the third post-fructose breath sample and the fourth post-fructose breath sample, and diagnosing the subject with fructose intolerance when the comparing meets a predetermined criteria.

Other embodiments provide a breath test method for screening for SIBO. The breath test screening method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a post-substrate breath sample from a subject, exposing the post-substrate breath sample to a sensor comprising hydrogen selective material having a resistivity that increases in response to increased presence of hydrogen, measuring resistivity of the post-substrate breath sample, comparing the measured resistivity of the baseline breath sample and the post-substrate breath sample and designating the subject has having an increased likelihood of having SIBO when comparing meets a predetermined criteria. The baseline breath sample can obtained after fasting for a time period and before ingesting the substrate. In some cases, the substrate can be a glucose-containing meal. In other cases, the substrate can be a lactulose-containing meal. The post-substrate breath sample can include a sample from the same subject after ingesting a substrate. The post-substrate breath sample can be obtained at a time period after ingesting the substrate. In some cases, the post-substrate breath sample is obtained at a time period of 20-30 minutes after ingesting the substrate. In other cases, the post-substrate breath sample is obtained at a time period of 30 minutes after ingesting the substrate.

In some embodiments, the step of collecting a post-substrate breath sample comprises collecting post-substrate breath samples at a plurality different time points. For example, in some cases, the plurality of time points include at least a first time point and a second time point. In other cases, the plurality of time points include at least a first time point, a second time point, a third time point and a fourth time point. In other cases, the plurality of time points include at least a first time point, a second time point, a third time point, a fourth time point and a fifth time point. In yet other cases, the plurality of time points include at least a first time point, a second time point, a third time point, a fourth time point, a fifth time point and a sixth time point. The time points can be spaced at any desired interval, such as a 15 minute interval, a 20 minute interval or a 30 minute interval. In certain cases, the first time point is a 15 minute time point, the second time point is a 30 minute time point, the third time point is a 45 minute time point, the fourth time point is a 60 minute time point, the fifth time point is a 75 minute time point and the sixth time point is a 90 minute time point. In other cases, the first time point is a 30 minute time point, the second time point is a 60 minute time point, the third time point is a 90 minute time point, the fourth time point is a 120 minute time point, the fifth time point is a 150 minute time point and the sixth time point is a 180 minute time point. Likewise, any desired number of time points can be used and the time points can be spaced by any desired time interval.

In some cases, the screening method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a first post-substrate breath sample from a subject at a first time point, exposing the first post-substrate breath sample to a sensor comprising hydrogen selective material having a resistivity that increases in response to increased presence of hydrogen, measuring resistivity of the first post-substrate breath sample, collecting a second post-substrate breath sample from a subject at a second time point, exposing the second post-substrate breath sample to a sensor comprising hydrogen selective material having a resistivity that increases in response to increased presence of hydrogen, measuring resistivity of the second post-substrate breath sample, comparing the resistivity of the baseline breath sample, first post-substrate breath sample and second post-substrate breath sample and designating the subject has having an increased likelihood of having SIBO when the comparing meets a predetermined criteria.

In some cases, the step of collecting a post-substrate breath sample comprises collecting a plurality of post-substrate breath samples at four time points. In such cases, the screening method includes steps of: collecting a baseline breath sample from a subject, exposing the baseline breath sample to a sensor comprising hydrogen selective material having a resistivity that increases in response to increased presence of hydrogen, measuring a resistivity of the sensor that occurs when the baseline breath sample contacts the sensor, prompting the subject to ingest a substrate, collecting a first post-substrate breath sample from a subject at a first time point, exposing the first post-substrate breath sample to the sensor, measuring a resistivity of the sensor that occurs when the first post-substrate breath sample contacts the sensor, collecting a second post-substrate breath sample from a subject at a second time point, exposing the second post-substrate breath sample to the sensor, measuring a resistivity of the sensor that occurs when the second post-substrate breath sample contacts the sensor, collecting a third post-substrate breath sample from a subject at a third time point, exposing the third post-substrate breath sample to the sensor, measuring a resistivity of the sensor that occurs when the third post-substrate breath sample contacts the sensor, collecting a fourth post-substrate breath sample from a subject at a second time point, exposing the fourth post-substrate breath sample to the sensor, measuring a resistivity of the sensor that occurs when the fourth post-substrate breath sample contacts the sensor, comparing the resistivity of the baseline breath sample, the first post-substrate breath sample, the second post-substrate breath sample, the third post-substrate breath sample and the fourth post-substrate breath sample and designating the subject has having an increased likelihood of having SIBO when the comparing meets a predetermined criteria.

In some cases, the breath test screening method for screening for SIBO is performed in connection with a breath analyzer. The breath analyzer can have any of the embodiments described herein. In some cases, the breath analyzer includes a portable, hand-held breath analyzer. The breath test diagnostic method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs when the baseline breath sample contacts the sensor, resetting the breath analyzer, prompting a subject to exhale a post-substrate breath sample into the breath analyzer, allowing the breath analyzer to measure a resistivity of the sensor that occurs when the post-substrate ingestion breath sample contacts the sensor, comparing the measured resistivity of the baseline breath sample to the measured resistivity of the post-substrate breath sample and designating the subject as having an increased likelihood of having SIBO when the comparing meets a predetermined criteria.

In other cases, the breath test screening method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-substrate breath sample into the breath analyzer, allowing the breath analyzer to measure a resistivity of the sensor that occurs with the first post-substrate ingestion breath sample, resetting the breath analyzer, prompting a subject to exhale a second post-substrate breath sample into the breath analyzer, allowing the breath analyzer to measure a resistivity of the sensor that occurs with the second post-substrate ingestion breath sample, comparing the measured resistivity of the baseline breath sample, the first post-substrate breath sample and the second post-substrate breath sample, and designating the subject as having an increased likelihood of having SIBO when the comparing meets a predetermined criteria.

In other cases, the breath test method for screening for SIBO includes the steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-substrate breath sample into the breath analyzer, allowing the breath analyzer to measure a resistivity of the sensor that occurs with the first post-substrate ingestion breath sample, resetting the breath analyzer, prompting a subject to exhale a second post-substrate breath sample into the breath analyzer, allowing the breath analyzer to measure a resistivity of the sensor that occurs with the second post-substrate ingestion breath sample, resetting the breath analyzer, prompting a subject to exhale a third post-substrate breath sample into the breath analyzer, allowing the breath analyzer to measure a resistivity of the sensor that occurs with the third post-substrate ingestion breath sample, resetting the breath analyzer, prompting a subject to exhale a fourth post-substrate breath sample into the breath analyzer, allowing the breath analyzer to measure a resistivity of the sensor that occurs with the fourth post-substrate ingestion breath sample, comparing the measured resistivity of the baseline breath sample, the first post-substrate breath sample, the second post-substrate breath sample, the third post-substrate breath sample and the fourth post-substrate breath sample, and designating the subject as having an increased likelihood of having SIBO when the comparing meets a predetermined criteria.

Other embodiments provide a breath test method for diagnosing SIBO. The breath test screening method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a post-substrate breath sample from a subject, exposing the post-substrate breath sample to a sensor comprising hydrogen selective material having a resistivity that increases in response to increased presence of hydrogen, measuring resistivity of the post-substrate breath sample, comparing the measured resistivity of the baseline breath sample and the post-substrate breath sample and diagnosing the subject with SIBO when the comparing meets a predetermined criteria.

In some cases, the step of collecting a post-substrate breath sample comprises collecting a plurality of post-substrate breath samples at different time points. In such cases, the diagnostic method includes steps of: collecting a baseline breath sample from a subject, exposing the baseline breath sample to a sensor comprising hydrogen selective material having a resistivity that increases in response to increased presence of hydrogen, measuring resistivity of the baseline breath sample, collecting a first post-substrate breath sample from a subject at a first time point, exposing the first post-substrate breath sample to a sensor comprising hydrogen selective material having a resistivity that increases in response to increased presence of hydrogen, measuring resistivity of the first post-substrate breath sample, collecting a second post-substrate breath sample from a subject at a second time point, exposing the second post-substrate breath sample to a sensor comprising hydrogen selective material having a resistivity that increases in response to increased presence of hydrogen, measuring resistivity of the second post-substrate breath sample, comparing the resistivity of the baseline breath sample, first post-substrate breath sample and second post-substrate breath sample and diagnosing the subject with SIBO when the comparing meets a predetermined criteria.

In some cases, the step of collecting a post-substrate breath sample comprises collecting a plurality of post-substrate breath samples at four time points. In such cases, the screening method includes steps of: collecting a baseline breath sample from a subject, exposing the baseline breath sample to a sensor comprising hydrogen selective material having a resistivity that increases in response to increased presence of hydrogen, measuring a resistivity of the sensor that occurs when the baseline breath sample contacts the sensor, prompting the subject to ingest a substrate-containing meal, collecting a first post-substrate breath sample from a subject at a first time point, exposing the first post-substrate breath sample to the sensor, measuring a resistivity of the sensor that occurs when the first post-substrate breath sample contacts the sensor, collecting a second post-substrate breath sample from a subject at a second time point, exposing the second post-substrate breath sample to the sensor, measuring a resistivity of the sensor that occurs when the second post-substrate breath sample contacts the sensor, collecting a third post-substrate breath sample from a subject at a third time point, exposing the third post-substrate breath sample to the sensor, measuring a resistivity of the sensor that occurs when the third post-substrate breath sample contacts the sensor, collecting a fourth post-substrate breath sample from a subject at a second time point, exposing the fourth post-substrate breath sample to the sensor, measuring a resistivity of the sensor that occurs when the fourth post-substrate breath sample contacts the sensor, comparing the resistivity of the baseline breath sample, the first post-substrate breath sample, the second post-substrate breath sample, the third post-substrate breath sample and the fourth post-substrate breath sample and diagnosing the subject with SIBO when the comparing meets a predetermined criteria.

In some cases, the breath test diagnostic method is performed in connection with a breath analyzer. The breath analyzer can have any of the embodiments described herein. In some cases, the breath analyzer includes a portable, hand-held breath analyzer. The breath test diagnostic method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs when the baseline breath sample contacts the sensor, resetting the breath analyzer, prompting a subject to exhale a post-substrate breath sample into the breath analyzer, allowing the breath analyzer to measure a resistivity of the sensor that occurs when the post-substrate breath sample contacts the sensor, comparing the measured resistivity of the baseline breath sample to the measured resistivity of the post-substrate breath sample and diagnosing the subject with SIBO when the comparing meets a predetermined criteria.

In other cases, the breath test diagnostic method includes steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-substrate breath sample into the breath analyzer, allowing the breath analyzer to measure a resistivity of the sensor that occurs with the first post-substrate breath sample, resetting the breath analyzer, prompting a subject to exhale a second post-substrate breath sample into the breath analyzer, allowing the breath analyzer to measure a resistivity of the sensor that occurs with the second post-substrate breath sample, comparing the measured resistivity of the baseline breath sample, the first post-substrate breath sample and the second post-substrate breath sample, and diagnosing the subject with SIBO when the comparing meets a predetermined criteria.

In other cases, the breath test diagnostic method includes the steps of: providing a breath analyzer having a sensor comprising hydrogen selective material having a resistivity that increases in response to increased presence of hydrogen, prompting a subject to exhale a baseline breath sample into the breath analyzer, allowing the breath analyzer to measure a resistivity of the sensor that occurs with the baseline breath sample, resetting the breath analyzer, prompting a subject to exhale a first post-substrate breath sample into the breath analyzer, allowing the breath analyzer to measure a resistivity of the sensor that occurs with the first post-substrate breath sample, resetting the breath analyzer, prompting a subject to exhale a second post-substrate breath sample into the breath analyzer, allowing the breath analyzer to measure a resistivity of the sensor that occurs with the second post-substrate breath sample, resetting the breath analyzer, prompting a subject to exhale a third post-substrate breath sample into the breath analyzer, allowing the breath analyzer to measure a resistivity of the sensor that occurs with the third post-substrate breath sample, resetting the breath analyzer, prompting a subject to exhale a fourth post-substrate breath sample into the breath analyzer, allowing the breath analyzer to measure a resistivity of the sensor that occurs with the fourth post-substrate breath sample, comparing the measured resistivity of the baseline breath sample, the first post-substrate breath sample, the second post-substrate breath sample, the third post-substrate breath sample and the fourth post-substrate breath sample, and diagnosing the subject with SIBO when the comparing meets a predetermined criteria.

In each of the above breath test methods, the baseline breath sample is a sample from a subject after fasting for at least 2 hours, 3 hours, 4 hours, 5 hours, 6 hours, 7 hours or 8 hours. In some cases, the baseline breath sample is a sample from a subject after fasting for at least 8 hours. Also, the step of resetting the breath analyzer comprises flushing the sensor with atmospheric air. In certain cases, the step of flushing the sensor with atmospheric air comprises opening an outlet in the sensor to release the breath sample, allowing the sensor to return to a baseline resistivity and closing the outlet. In other embodiments, the step of resetting the breath analyzer comprises replacing the sensor. In some cases, the step of replacing the sensor occurs each time a breath sample is measured.

Various examples of the invention have been described. Although the present invention has been described in considerable detail with reference to certain disclosed embodiments, the embodiments are presented for purposes of illustration and not limitation. Other embodiments incorporating the invention are possible. One skilled in the art will appreciate that various changes, adaptations, and modifications may be made without departing from the spirit of the invention and the scope of the appended claims.

What is claimed is:

1. A handheld, portable breath analyzer, comprising:
a main body; and
a removable mouthpiece, wherein the removable mouthpiece removably attaches to the main body;
wherein the main body includes a sensor, a processor, a power source and an electrical circuit;
wherein the electrical circuit operably connects the power source to the sensor and connects the sensor to the processor;
wherein the sensor comprises a conductive material and hydrogen selective material in contact with the conductive material, wherein the hydrogen selective material has a resistivity that increases in response to increased concentration of hydrogen; and
wherein the processor detects resistivity of the sensor and uses the resistivity to calculate a concentration of hydrogen.

2. The breath analyzer of claim 1 wherein the hydrogen selective material comprises polyaniline, wherein the polyaniline is doped with a dopant that increases pH sensitivity of the polyaniline, and wherein the polyaniline has a resistivity that increases in response to increased concentration of hydrogen.

3. The breath analyzer of claim 2 wherein the polyaniline has a pH sensitivity of more than 59 mV.

4. The breath analyzer of claim 2 wherein the dopant comprises a protonic acid.

5. The breath analyzer of claim 4 wherein the dopant comprises a protonic acid selected from the group consisting of hydrochloric acid, sulfuric acid, salicylic acid, acetic acid, citric acid, tartaric acid, oxalic acid, malonic acid, succinic acid, glutamic acid, adipic acid, phthalic acid and camphor sulfonic acid.

6. The breath analyzer of claim 5 wherein the dopant consists essentially of hydrochloric acid.

7. The breath analyzer of claim 1 wherein the conductive comprises a plurality of electrodes.

8. The breath analyzer of claim 7 wherein the plurality of interdigitated finger electrodes.

9. The breath analyzer of claim 1 wherein the hydrogen selective material is deposited as a thin film on the conductive material.

10. The breath analyzer of claim 1 further comprising a desiccant or gas filter, wherein the desiccant or gas filter is located in either the removable mouthpiece or the main body.

11. A breath test method for screening for a gastrointestinal disorder, comprising steps of:
(a) providing a portable, hand-held breath analyzer, wherein the portable, hand-held breath analyzer comprises:
(i) a main body; and
(ii) a removable mouthpiece, wherein the removable mouthpiece removably attaches the main body, wherein the main body includes a sensor, a processor, a power source and an electrical circuit, wherein the electrical circuit operably connects the power source to the sensor and connects the sensor to the processor; wherein the sensor comprises a conductive material and hydrogen selective material in contact with the conductive material, wherein the hydrogen selective material has a resistivity that increases in response to increased concentration of hydrogen;
(b) prompting a subject to exhale a breath sample into the removable mouthpiece;
(c) allowing the processor to measure a resistivity of the sensor that occurs when the breath sample contacts the sensor; and
(f) designating the subject as having an increased likelihood of having a gastrointestinal disorder if the measured resistivity is above and/or beneath a predetermined value.

12. The breath test method of claim 11 wherein the gastrointestinal disorder is celiac disease.

13. The breath test method of claim 11 wherein the gastrointestinal disorder is non-celiac gluten sensitivity.

14. A breath test method for diagnosing a gastrointestinal disorder, comprising steps of:
(a) providing a portable, hand-held breath analyzer, wherein the portable, hand-held breath analyzer comprises:
(i) a main body; and
(ii) a removable mouthpiece, wherein the removable mouthpiece removably attaches the main body, wherein the main body includes a sensor, a processor, a power source and an electrical circuit, wherein the electrical circuit operably connects the power source to the sensor and connects the sensor to the processor; wherein the sensor comprises a conductive material and hydrogen selective material in contact with the conductive material, wherein the hydrogen selective material has a resistivity that increases in response to increased concentration of hydrogen;
(b) prompting a subject to exhale a breath sample into the removable mouthpiece;
(c) allowing the processor to measure a resistivity of the sensor that occurs when the breath sample contacts the sensor; and
(f) diagnosing the subject as having a gastrointestinal disorder if the measured resistivity is above and/or beneath a predetermined value.

15. The breath test method of claim 14 wherein the gastrointestinal disorder is celiac disease.

16. The breath test method of claim 14 wherein the gastrointestinal disorder is non-celiac gluten sensitivity.

17. A breath test method for screening for lactose intolerance, comprising steps of:
(a) providing a portable, hand-held breath analyzer, wherein the portable, hand-held breath analyzer comprises:
(i) a main body; and
(ii) a removable mouthpiece, wherein the removable mouthpiece removably attaches the main body, wherein the main body includes a sensor, a processor, a power source and an electrical circuit, wherein the electrical circuit operably connects the power source to the sensor and connects the sensor to the processor; wherein the sensor comprises a conductive material and hydrogen selective material in contact with the conductive material, wherein the hydrogen selective material has a resistivity that increases in response to increased concentration of hydrogen;
(a) prompting a subject to exhale a baseline breath sample into the removable mouthpiece;
(b) allowing the processor to measure a resistivity of the sensor that occurs when the baseline breath sample contacts the sensor;
(c) prompting a subject to ingest a lactose-containing meal;
(d) prompting a subject to exhale a post-lactose breath sample into the removable mouthpiece;
(e) allowing the processor to measure a resistivity of the sensor that occurs when the post-lactose breath sample contacts the sensor;
(f) comparing the measured resistivity of the baseline breath sample to the measured resistivity of the post-lactose breath sample;
(g) designating the subject as having an increased likelihood of having lactose intolerance if the comparing meets a predetermined criterion.

18. The breath test method of claim 16 wherein the designating the subject as having an increased likelihood of having lactose intolerance occurs when the measured resistivity is above and/or beneath a predetermined value.

19. The breath test method of claim 17, comprising:
(a) prompting a subject to exhale a first post-lactose breath sample into the removable mouthpiece at a first time point;
(e) allowing the processor to measure a resistivity of the sensor that occurs when the first post-lactose breath sample contacts the sensor;
(a) prompting a subject to exhale a second post-lactose breath sample into the removable mouthpiece at a second time point;
(e) allowing the processor to measure a resistivity of the sensor that occurs when the second post-lactose breath sample contacts the sensor;
(f) comparing the measured resistivity of the baseline breath sample, the first post-lactose breath sample and the second post-lactose breath sample;
(g) designating the subject as having an increased likelihood of having lactose intolerance if the comparing meets a predetermined criterion.

20. The breath test method of claim 19, comprising:
(a) prompting a subject to exhale a first post-lactose breath sample into the removable mouthpiece at a first time point;
(b) allowing the processor to measure a resistivity of the sensor that occurs when the first post-lactose breath sample contacts the sensor;
(c) prompting a subject to exhale a second post-lactose breath sample into the removable mouthpiece at a second time point;
(d) allowing the processor to measure a resistivity of the sensor that occurs when the second post-lactose breath sample contacts the sensor;
(e) prompting a subject to exhale a third post-lactose breath sample into the removable mouthpiece at a third time point;
(f) allowing the processor to measure a resistivity of the sensor that occurs when the third post-lactose breath sample contacts the sensor;
(g) prompting a subject to exhale a fourth post-lactose breath sample into the removable mouthpiece at a fourth time point;
(h) allowing the processor to measure a resistivity of the sensor that occurs when the fourth post-lactose breath sample contacts the sensor;
(i) comparing the measured resistivity of the baseline breath sample, the first post-lactose breath sample, the second post-lactose breath sample, the third post-lactose breath sample and the fourth post-lactose breath sample; and
(j) designating the subject as having an increased likelihood of having lactose intolerance if the comparing meets a predetermined criterion.

21. The breath test method of claim 20 wherein the first time point is a 30 minute time point, the second time point is a 60 minute time point, the third time point is a 90 minute time point and the fourth time point is a 120 minute time point.

22. A breath test method for screening for fructose intolerance, comprising steps of:
(a) providing a portable, hand-held breath analyzer, wherein the portable, hand-held breath analyzer comprises:
(i) a main body; and
(ii) a removable mouthpiece, wherein the removable mouthpiece removably attaches the main body, wherein the main body includes a sensor, a processor, a power source and an electrical circuit, wherein the electrical circuit operably connects the power source to the sensor and connects the sensor to the processor; wherein the sensor comprises a conductive material and hydrogen selective material in contact with the conductive material, wherein the hydrogen selective material has a resistivity that increases in response to increased concentration of hydrogen;
(a) prompting a subject to exhale a baseline breath sample into the removable mouthpiece;
(b) allowing the processor to measure a resistivity of the sensor that occurs when the baseline breath sample contacts the sensor;
(c) prompting a subject to ingest a fructose-containing meal;
(d) prompting a subject to exhale a post-fructose breath sample into the removable mouthpiece;
(e) allowing the processor to measure a resistivity of the sensor that occurs when the post-fructose breath sample contacts the sensor;
(f) comparing the measured resistivity of the baseline breath sample to the measured resistivity of the post-fructose breath sample;
(g) designating the subject as having an increased likelihood of having fructose intolerance if the comparing meets a predetermined criterion.

23. The breath test method of claim 22 wherein the designating the subject as having an increased likelihood of having fructose intolerance occurs when the measured resistivity is above and/or beneath a predetermined value.

24. The breath test method of claim 22, comprising:
(a) prompting a subject to exhale a first post-fructose breath sample into the removable mouthpiece at a first time point;
(e) allowing the processor to measure a resistivity of the sensor that occurs when the first post-fructose breath sample contacts the sensor;
(a) prompting a subject to exhale a second post-fructose breath sample into the removable mouthpiece at a second time point;
(e) allowing the processor to measure a resistivity of the sensor that occurs when the second post-fructose breath sample contacts the sensor;
(f) comparing the measured resistivity of the baseline breath sample, the first post-fructose breath sample and the second post-fructose breath sample;
(g) designating the subject as having an increased likelihood of having fructose intolerance if the comparing meets a predetermined criterion.

25. The breath test method of claim 24, comprising:
(a) prompting a subject to exhale a first post-fructose breath sample into the removable mouthpiece at a first time point;
(b) allowing the processor to measure a resistivity of the sensor that occurs when the first post-fructose breath sample contacts the sensor;
(c) prompting a subject to exhale a second post-fructose breath sample into the removable mouthpiece at a second time point;
(d) allowing the processor to measure a resistivity of the sensor that occurs when the second post-fructose breath sample contacts the sensor;
(e) prompting a subject to exhale a third post-fructose breath sample into the removable mouthpiece at a third time point;
(f) allowing the processor to measure a resistivity of the sensor that occurs when the third post-fructose breath sample contacts the sensor;
(g) prompting a subject to exhale a fourth post-fructose breath sample into the removable mouthpiece at a fourth time point;
(h) allowing the processor to measure a resistivity of the sensor that occurs when the fourth post-fructose breath sample contacts the sensor;
(i) comparing the measured resistivity of the baseline breath sample, the first post-fructose breath sample, the second post-fructose breath sample, the third post-fructose breath sample and the fourth post-fructose breath sample; and
(j) designating the subject as having an increased likelihood of having fructose intolerance if the comparing meets a predetermined criterion.

26. The breath test method of claim 25 wherein the first time point is a 30 minute time point, the second time point is a 60 minute time point, the third time point is a 90 minute time point and the fourth time point is a 120 minute time point.
